(19) United States
(12) Patent Application Publication
(10) Pub. No.: US 2014/0243422 A1
(43) Pub. Date: Aug. 28, 2014

(54) METHODS OF TREATING BEHAVIORAL AND PSYCHIATRIC DISORDERS

(71) Applicants: Susan Abushakra, San Jose, CA (US); Gerald Crans, Redwood City, CA (US); Ramon Hernandez, San Carlos, CA (US); Jesse Cedarbaum, Woodbridge, CT (US)

(72) Inventors: Susan Abushakra, San Jose, CA (US); Gerald Crans, Redwood City, CA (US); Ramon Hernandez, San Carlos, CA (US); Jesse Cedarbaum, Woodbridge, CT (US)

(73) Assignee: ELAN PHARMACEUTICALS, INC., CAMBRIDGE, MA (US)

(21) Appl. No.: 14/123,581

(22) PCT Filed: Jun. 4, 2012
(86) PCT No.: PCT/US2012/040789

§ 371 Date: May 8, 2014

Related U.S. Application Data
(60) Provisional application No. 61/520,031, filed on Jun. 3, 2011.
(60) Provisional application No. 61/541,333, filed on Sep. 30, 2011.
(60) Provisional application No. 61/618,680, filed on Mar. 31, 2012.

(51) Int. Cl. A61K 31/047

(52) U.S. Cl. CPC A61K 31/047
USPC 514/729; 568/833

(57) ABSTRACT

The invention relates to the treatment of disorders associated with elevated myo-inositol levels in brain, in particular behavioural and neuropsychiatry disorders such as dementia, mild Alzheimer's disease, mild cognitive impairment or bipolar disorder by administering an effective amount of scyllo-inositol to a subject.

### FIELD OF THE INVENTION

The invention relates to methods for treating disorders associated with elevated myo-inositol levels in brain, and in particular behavioural and neuropsychiatric disorders.

The term “Behavioral and Psychological Symptoms in Dementia” (BPSD) has been coined to describe the spectrum of behavioral disturbances or neuropsychiatric symptoms (NPS) that are Important manifestations of long-term progressive neurodegenerative processes in resulting in what is known clinically as dementia. The BPSD umbrella term encompasses a wide spectrum of NPS which include apathy or indifference, affective and psychotic symptoms, disinhibition and hyperactivity, irritability, agitation/aggression, changes in appetite, and night time confusion or sleep disturbances. The various combinations of symptoms (sub-syndromes) of BPSD occur in most of the dementias. These dementias include Alzheimer's disease dementia (AD), Fronto-temporal Dementia (FTD), Vascular dementia, Lewy Body disease (LBD), and Downs dementia. Although the BPSD are not disease specific, there are certain profiles of neuropsychiatric disturbances that are characteristic of specific diseases.

For example, in AD, apathy and affective symptoms (affect refers to the experience of feeling or emotion, and is often referred to as blunted or flat in apathy and depression) are common early in the disease whereas psychotic symptoms, aberrant motor behavior, and disinhibition occur late in the course of the dementias. In FTD, apathy/indifference, social disinhibition, and personality changes occur very early, and may even be the presenting manifestations (for example, in behavioral variant FTD (bv-FTD). LBD is characterized by fluctuating cognition, extrapyramidal motor symptoms, diurnal rhythm disturbances, visual hallucinations, nighttime agitation and depression. Vascular dementia is characterized by marked apathy, lack of initiative, irritability and depression.

Unlike the progressive decline in memory, reasoning, and language skills in AD, some NPS (such as depression and anxiety) may have a fluctuating course with some symptoms remitting and others emerging at different stages of the disease. However, as the disease advances the apathy, agitation, and hyperactivity symptoms become more persistent and progressive, leading to a heavy burden of psychopathology. Some of the NPS, such as agitation/aggression, hallucinations or delusions, aberrant motor behaviors, and disinhibition, are especially difficult to manage and represent a major source of caregiver distress. The psychotic symptoms and agitation/aggression are frequent causes of nursing home placement in moderate to severe AD. The BPSD associated with Alzheimer's disease are the most well-studied of these neuropsychiatric syndromes.

BPSD Symptom Clusters or Sub-Syndromes

In order to facilitate neurobiological studies and therapeutic trials of BPSD, the NPS have been grouped into symptom clusters. These clusters were based on either latent class or factor analyses in dementia populations. Despite methodological differences, including sample size and use of population-based or clinic-based samples, these studies have consistently identified either 3 or 4 clusters or sub-syndromes. The most consistent clusters are: i) affective, ii) psychotic, and iii) hyperactivity sub-syndromes. The core symptoms for the affective sub-syndrome are depression and anxiety, but some studies include irritability and agitation in this cluster. The psychotic cluster consistently includes delusions and hallucinations; while the hyperactivity cluster usually includes aberrant motor behavior, elation and disinhibition, and is considered a frontal lobe or “dysexecutive syndrome”. It remains a matter of debate whether apathy is part of the affective cluster or is a separate syndrome. Appetite and sleep changes (or night time behaviors) are frequently associated with affective symptoms, but can also be related to a patient's comorbid medical conditions. The utility of the cluster approach is that it helps define clinical syndromes that may share a common neurochemical basis and may potentially respond to similar classes of drugs. This syndromic approach to BPSD also facilitates the recognition and accurate diagnosis of these sub-syndromes present in the dementias, and may inform medical professionals of their appropriate management.

Epidemiology of BPSD

The prevalence of BPSD in the Alzheimer's Disease (AD) population is estimated to be 60-90%, depending on study methodology, with a life time risk approaching 100%. The prevalence and number of NPS in AD is known to increase with disease severity and duration. An increase in the prevalence of NPS is associated with progression from Mild Cognitive Impairment (MCI) to AD.

The 2 earliest NPS to emerge in the progression of AD are usually apathy or depression, either of which may manifest at the mild cognitive impairment (MCI) stage or early AD stage. Apathy remains highly prevalent throughout the course of disease, and typically follows a progressive course. In contrast, the severity of depression or dysphoria tends to fluctuate especially early in the disease. Depression has high prevalence in mild and moderate AD, but becomes less prevalent in severe AD. Anxiety has a somewhat lower prevalence than depression but tends to follow a course similar to, and is frequently associated with depression in AD. The other symptoms that occur with medium to high prevalence in mild or moderate disease are Irritability, agitation/aggression, and appetite changes (Aalten et al 2007, Neuropsychiatric syndromes in dementia. Results from the European Alzheimer Disease Consortium: Part 1. Dement Geriatr Cogn Disord 2007; 24:457-63 and, Steinberg et al 2008, Point and 5-year period prevalence of neuropsychiatric symptoms in dementia: The Cache County Study. International Journal of Geriatric Psychiatry, 23(2): 170-177). Elation or euphoria are among the least common NPS, but may be seen in association with disinhibition and aberrant motor behavior as part of the frontal lobe or dysexecutive syndrome. The hyperactivity symptoms (aberrant motor behavior, nighttime behaviors, and disinhibition) become relatively common in moderate to severe AD. Delusions and hallucinations, which are uncommon at the early and mild stage, also become more prevalent in the severe stages of AD. The cumulative burden of behavioral dysfunction with advancing AD becomes significant and eventually poses a significant management challenge in AD.

Neurobiological Basis of BPSD Syndromes

The various NPS in dementia can be viewed as an expression of the regional degenerative changes that are specific to each dementia. Since brain regions demonstrate selective vulnerability to various misfolded protein pathologies, the earliest NPS to emerge in a dementia disorder will depend on the site of selective vulnerability. The neurochemical basis of apathy, depression, agitation and psychotic symptoms has been elucidated using increasingly sophisticated methodologies. These have included receptor binding, functional/volumetric imaging, genomic associations, and autopsy studies.

In AD, the amyloid pathology usually starts in the entorhinal and parietal cortex, but frontal lobe pathology is also seen and may present as apathy or decreased initiative, reflecting decreased basal forebrain cholinergic activity. Apathy has also been shown to correlate with the amount of neurofibrillary tangles (aggregates of hyper-phosphorylated tau protein also referred to as NFT) in autopsy studies. The early appearance of affective symptoms is thought to reflect dysfunction of various monoaminergic networks with significant reduction of noradrenergic and serotonergic levels in locus ceruleus and dorsal raphe nuclei, respectively, and with decreased substantia nigra dopamine levels. Functional imaging studies suggest hypometabolism bilaterally in the anterior cingulate, and in superior temporal and in superior frontal lobes.

Agitation and aberrant motor behavior, which become prominent in advanced AD were shown to be related to NFT load in the orbitofrontal cortex. The appearance of psychotic symptoms has been associated with worse cognitive and functional outcomes. Delusions and hallucinations were found to correlate with M2 subtype, but not M1, muscarinic receptor density in the orbitofrontal amd mid-temporal cortex respectively. Together with agitation, psychotic symptoms were also associated with specific dopamine receptor gene variations. In addition, polymorphisms of the serotonin transporter and 5-HT2A receptor polymorphisms were associated with agitation/aggression and psychosis respectively.

In the Vascular dementia caused by subcortical ischemic vascular disease, there is early involvement of frontal white matter causing de-afferentation (elimination or interruption of afferent nerve impulses) and cholinergic loss in the basal forebrain. Early disruption of the mesial prefrontal network and its subcortical connections leads to apathy and loss of drive, while disruption of the dorsolateral prefrontal network manifests as executive dysfunction; both these symptoms are early manifestations of Vascular dementia. The later involvement of the ventral orbitofrontal network results in appearance of socially inappropriate and/or dis-inhibited behaviors.

Behavioral variant FTD (bv-FTD) is characterized by early frontal and anterior temporal atrophy. The BPSD symptoms of bv-FTD reflect cholinergic loss in the frontal lobes and functional deficits in the “salient network” with appearance of emotional blunting, lack of empathy and disinhibition. LBD is described to manifest widespread dopaminergic and cholinergic loss which affects the cortex diffusely. The BPSD in LBD pose a unique treatment challenge because of the marked sensitivity of these patients to anti-cholinergic effects and especially to neuroleptic medications.

Current Treatments Options and Unmet Medical Need

There are currently no drugs approved for the long term management of BPSD. Risperidone (Risperidal®), an atypical anti-psychotic, is the only drug approved for the short term management of severe agitation. This approval was limited to some European countries, and was based on 2 positive controlled trials of short duration, but not all trials were consistently positive. The major drug classes that have been studied in BPSD are: the atypical antipsychotics including risperidone, quetiapine (Seroquel®) and ziprasidone (Geodon®); the drugs that treat symptoms of cognitive decline in AD, (cholinesterase inhibitors and memantine); and antidepressants (such as the selective serotonin reuptake inhibitors (SSRIs) or the serotonin norepinephrine reuptake inhibitors (SNRIs)). The atypical antipsychotic studies focused on AD patients with psychotic or agitation/aggression symptoms. The results of these studies were not consistently positive, and a comparative trial of 3 drugs vs placebo showed that low tolerability was also a limitation. In addition, the use of these drugs in AD patient is associated with increased risk of mortality. This led the US FDA to issue a safety warning for older dementia patients of all the atypical antipsychotics and to place “boxed warning” in the drug labels stating the increased risk of death and that these drugs are not approved by the Food and Drug Administration (FDA) for the treatment of behavior problems in older adults with dementia. There have been few studies with acetyl cholinesterase inhibitors (ChEI) where behavioral symptoms were measured as the primary outcome, and meta-analyses of ChEI trials have not shown consistent effects on NPS. Some studies have shown effects on apathy, while others have not, some have shown some benefit on affective symptoms. The ChEI effects are thought to be modest and of limited clinical relevance. Studies of antidepressants and mood stabilizers have not shown consistent benefit across trials on non-depression-related NPS; one study with citalopram (Celexa®) showed a possible benefit on agitation and emotional labililty only. A meta-analysis of studies targeting depression in AD suggested a modest benefit with second generation antidepressants (SSRIs/SNRIs), and the study also found they were associated with higher discontinuation rates due to adverse events in this aged patient population. The pharmacological treatment choices for BPSD are therefore quite limited and leave an unmet need for a safe, well-tolerated drug with beneficial clinical effects on the behavioral and psychiatric symptoms.

Lithium (administered as lithium carbonate and abbreviated herein as Li or LI), a mood stabilizing agent, is known to decrease brain myo-inositol levels. Of note, lithium treatment was shown to induce early reduction of myo-inositol levels by ˜30% in bipolar patients (within a few days), followed 2 weeks later by improved affective symptoms. The observed delay between myo-inositol reduction and improved affect suggests that the early Li-induced alteration of phospho-inositide signaling (phospho-inositides comprise a myo-inositol molecule bonded through the alcohol functionality to one or more phosphate groups and are also called second messengers that modulate other cell processes is likely to lead to downstream effects on gene expression and protein synthesis that mediate its clinical effect on mood.

Over the last 10 years, the medical community and health agencies have become more aware of the importance of BPSD, and of the need to develop management strategies and effective treatments to minimize its impact. The US FDA organized an advisory meeting in collaboration with the American Association of Geriatric Psychiatry in March 2000 to discuss this topic and to focus attention on the need for research and drug development efforts in this area (T. Laughren, 2001, Am J Geriatr Psychiatry, 9(4):340-5). The FDA accepted “BPSD” as a broad concept that encompasses the various behavioral manifestations of dementia. The agency emphasized the need to identify more specific sub-syndromes (or clusters of symptoms) that are likely to share a common neurochemical basis. Such sub-syndromes could then be specifically targeted by drugs with the appropriate mechanism of action. At the time the agency accepted the diagnostic validity of the “Psychosis of AD” based on criteria that were published shortly before the consensus meeting (Jeste and Finkel, 2000 μm J Gerlatr Psychiatry, 8(1):29-34). Since then, provisional diagnostic criteria have been published for “Depression in AD” (Olin et al 2002, Geriatric Psychiatry, 10, 125-128) and for Apathy as a stand-alone syndrome (Robert et al 2009, Eur Psychiatry, 24(2):98-104). However, no drugs have been approved for these specific indications.

Several scales have been used for the assessment of BPSD. These include scales that have been specifically designed for studying NPS in patients with dementia, such as the “Behavioral Pathology in AD Rating Scale” (BEHAVE-AD, Reisberg et al. BEHAVE-AD: A clinical rating scale for the assessment of pharmacologically remediable behavioral symptomatology in Alzheimer's disease. In: Altman H J, editor. Alzheimer's Disease: Problems, prospects, and perspectives. Plenum; New York: 1987. pp. 1-161996), CERAD behavior rating scale for dementia (BRSD, Tariot 1996, International Psychogeriatrics, 8(Suppl. 3):317-320), and the Neuropsychiatric Inventory (NPI, Cummings et al. 1994, Neurology. 1994; 44:2308-14). The NPI which was designed to evaluate the broad range of behavioral symptoms that may develop in dementia patients, is a well validated instrument and has been widely used in BPSD studies and in AD treatment trials. The original version assessed 10 symptoms or items, including: delusions, hallucinations, agitation/aggression, depression/dysphoria, anxiety, apathy, irritability, euphoria/elation, disinhibition, and aberrant motor behavior; and was subsequently updated to include appetite changes and nighttime behaviors (sleep disturbances). The NPI is administered by a trained individual as a structured interview with the patient's caregiver. Each item is assessed by a scripted screening question, and the frequency and severity of each item is scored from 0-4 (0: absent, 1: occasional or less than once per week to 4 occurs at least daily), and from 1-3 (1: mild or produces little distress in patient; 2: moderate or more disturbing to patient but can be re-directed by caregiver; and 3: severe or very disturbing to patient and difficult to re-direct). The total item-score is then derived from frequency multiplied by the severity score; and the total NPI score (NPI-T) is derived from addition of all item scores, with the range of scores being from 0 (no NPS at all) to 144 (all 12 NPS are present at maximum severity at daily basis). In the majority of the drug trials in Mild/Moderate AD, the mean total NPI scores are ˜10-12 at baseline. The Phase 2 study described in the example below utilized the NPI-12 item assessment; and the mean total NPI scores were between 8 and 10 at baseline. This baseline severity is slightly lower than the range reported in many Mild to Moderate AD trials reported in the literature.

It is to be understood that both the foregoing general description and the following detail description are exemplary only and are not restrictive of the disclosure, as claimed.

### SUMMARY OF THE INVENTION

It has been unexpectedly discovered, that administration of scyllo-inositol to a patient down regulates (i.e. reduces) the level of myo-inositol in the brain in such patients and delays the emergence, and lessens the degree, of neuropsychiatric symptoms (NPS). Accordingly, in an aspect of the invention, there is provided a method of reducing the level of myo-inositol in a subject's brain comprising administering an effective amount of scyllo-insolitol to the subject. In aspects, the invention provides scyllo-inositol or a pharmaceutical composition comprising scyllo-inositol for use in reducing the level of, or down-regulating myo-inositol in a subject's brain or in the preparation of a medicament for reducing the level of, or down-regulating myo-inositol in a subject's brain.

Methods of the invention may be used to reduce levels of or down-regulate myo-inositol in patients suffering from dementia, mild AD, MCI, or bipolar disorder. Therefore, in an aspect of the invention, a method is provided for down-regulating myo-inositol in a dementia, mild AD, MCI or bipolar patient's brain comprising administering an effective amount of scyllo-inositol or a composition comprising scyllo-inositol for a therapeutically effective treatment period wherein the administration of scyllo-inositol or composition reduces the levels in a patient's brain from a baseline measurement taken prior to administration.

In embodiments of the invention, the level of myo-inositol is reduced by less than about 60%. In embodiments of the invention the level of myo-inositol is reduced by about 20 to about 55%. In embodiments of the invention the level of myo-inositol is reduced by about 20 to about 50%. In other embodiments of the invention the level of myo-inositol is reduced by about 25% to about 45%. In further embodiments the level of myo-inositol is reduced by about 25% to about 35%.

In another aspect of the invention, there is provided a method for treating neuropsychiatric symptoms in a subject comprising administering an effective amount of scyllo-inositol to the subject. In other aspects, the invention relates to scyllo-inositol or a composition comprising scyllo-inositol for use in the treatment of neuropsychiatric symptoms in a subject or in the preparation of a medicament for treatment of neuropsychiatric symptoms in a subject.

An aspect the invention provides a method for delaying emergence and/or decreasing severity of neuropsychiatric symptoms or a neuropsychiatric symptom cluster in a patient comprising: administering to the patient an effective amount of scyllo-inositol or a pharmaceutical composition comprising an effective amount of scyllo-inositol. In embodiments of the invention, the neuropsychiatric symptoms are affective, behavioral, frontal or apathetic symptoms. In embodiments of the invention, the neuropsychiatric symptoms comprise at least two affective, behavioral, frontal or apathetic symptoms. In particular embodiments the neuropsychiatric symptoms are selected from the group consisting of, or chosen from, depression, anxiety, appetite change, agitation, nighttime behavior, delusions, hallucinations, apathy, irritability, aberrant motor behavior, disinhibition, sleep disturbances and elation. In particular embodiments the neuropsychiatric symptoms are selected from the group consisting of, or chosen from, depression, anxiety, appetite change, agitation, nighttime behavior, delusions, hallucinations, apathy, disinhibition, sleep disturbances and elation. In particular embodiments the neuropsychiatric symptoms are selected from the group consisting of, or chosen from, depression, anxiety, appetite change, agitation and apathy. In particular embodiments, the neuropsychiatric symptoms are selected from the group consisting of, or chosen from, disinhibition, sleep disturbances, apathy and elation. In an embodiment of the invention, the neuropsychiatric symptom cluster is selected from the group consisting of, or chosen from, affective cluster, psychotic cluster, apathy, frontal lobe elation and disinhibition cluster, behavioral cluster, and any combination thereof.

In an aspect the invention provides a method for delaying the emergence of at least two new neuropsychiatric symptoms in a patient comprising: administering to the patient an effective amount of scyllo-inositol or a pharmaceutical composition comprising an effective amount of scyllo-inositol over a therapeutically effective treatment period wherein over the treatment period, the administration of scyllo-inositol delays the emergence of at least two new neuropsychiatric symptoms compared to a baseline measurement prior to administration. The patient may be suffering from dementia, in particular Alzheimer's disease dementia, fronto-temporal dementia, vascular dementia, Lewy Body dementia, and Downs dementia. In embodiments of the invention, the patient is suffering from Alzheimer's disease dementia and has mild or moderate Alzheimer's disease.

In an aspect the invention provides a method for delaying the emergence of at least one neuropsychiatric symptom cluster in a patient with a dementia comprising: administering to the patient an effective amount of scyllo-inositol or a pharmaceutical composition comprising an effective amount of scyllo-inositol over a therapeutically effective treatment period, wherein over the treatment period, the administration of scyllo-inositol or composition delays the emergence of at least one neuropsychiatric symptom cluster compared to a baseline measurement prior to administration. In an embodiment, the neuropsychiatric symptom cluster is chosen from affective cluster, psychotic cluster, apathy, frontal lobe elation and disinhibition cluster, behavioral cluster, and any combination thereof. In an embodiment, the dementia is chosen or selected from the group consisting of Alzheimer's disease dementia, fronto-temporal dementia, vascular dementia, Lewy Body dementia, and Downs dementia. In a particular embodiment, the dementia is moderate Alzheimer's disease and the at least one cluster is behavioral.

In an aspect the Invention provides a method for reducing the severity of at least one neuropsychiatric symptom in a patient with dementia comprising: administering to the patient an effective amount of scyllo-inositol or a pharmaceutical composition comprising an effective amount of scyllo-inositol over a therapeutically effective treatment period, wherein over the treatment period, the administration of scyllo-inositol reduces the severity of at least one neuropsychiatric symptom from a baseline measurement prior to administration. In an embodiment, the at least one neuropsychiatric symptom is selected from the group consisting of, or chosen, from depression, anxiety, appetite change, agitation, apathy, disinhibition, sleep disturbances, apathy and elation. In an embodiment, the dementia is chosen or selected from the group consisting of Alzheimer's disease dementia, fronto-temporal dementia, vascular dementia, Lewy Body dementia, and Downs dementia.

In aspects of the invention, the treatment period is at least about 12 weeks, at least about 24 weeks, at least about 48 weeks or at least about 78 weeks. In an embodiment of the invention the treatment period is at least about 48 weeks. In another embodiment of the invention the treatment period is at least about 78 weeks.

In an aspect, the invention provides a method for delaying the progression of at least one existing neuropsychiatric symptom in a patient with a dementia comprising: administering to the patient an effective amount of scyllo-inositol or a pharmaceutical composition comprising an effective amount of scyllo-inositol over a treatment period of at least 78 weeks, wherein over the treatment period, the administration of scyllo-inositol delays the progression of at least one existing neuropsychiatric symptom from a baseline measurement prior to administration. In an embodiment the dementia is selected from the group consisting of or chosen from Alzheimer's disease dementia, fronto-temporal dementia, vascular dementia, Lewy Body dementia, and Downs dementia. In a particular embodiment, the dementia is moderate or severe Alzheimer's disease.

In particular embodiments of the invention, the effective amount of scyllo-inositol is about 250 mg. In particular embodiments of the invention, scyllo-inositol or the pharmaceutical composition comprising scyllo-inositol is administered twice a day.

### BRIEF DESCRIPTION OF THE DRAWINGS

FIG. 1 is a chart of the proportion of patients who developed new NPS over the 78 weeks of the study shown for the Mild m-ITT population (MMSE: 22-26).

FIGS. 2A and B are plots of the prevalence of various NPI symptoms at baseline and at week 78: Placebo (FIG. 2A) and 250 mg group (FIG. 2B).

FIG. 3 is a plot of the prevalence of newly emergent NPI symptoms at any time during the study shown in order of decreasing prevalence in the placebo group (Mild MMSE: 22-26).

FIG. 4 is a chart of the proportion of patients who developed new NPS over the 78 weeks of the study shown for the Moderate m-ITT population (MMSE: 16-21).

FIG. 5 is a plot of the prevalence of newly emergent NPI symptoms at any time during the study shown in order of decreasing prevalence in the placebo group (Moderate MMSE: 16-21).

FIG. 6 is a chart of the effects of scyllo-inositol on myo-inositol and scyllo-inositol brain levels by MR spectroscopy (myo-inositol on left, scyllo-inositol on right).

FIG. 7 is a plot of the correlations between maximal drug concentrations (Cmax) in Plasma, brain, and CSF and the CSF Abeta42/40 ratio. A lower ratio (i.e. Abeta42, the more fibrillogenic form, is less prevalent) is postulated to be clinically beneficial.

FIGS. 8A and 8B is a chart of the relationships between scyllo-inositol plasma exposures (Quartiles of Plasma AUC) and probability of emergence of affective symptoms.

FIG. 9 are four plots of MRS brain scan showing levels of scyllo-inositol and myo-inositol at baseline (labeled Screening) and 24 weeks for 250 mg bid and 1000 mg bid doses.

### DETAILED DESCRIPTION OF THE INVENTION

Particular aspects of the disclosure are described in greater detailed below. The terms, definitions and abbreviations as used in the present application and as clarified or designated herein are intended to represent the meaning within the present disclosure. The patent and scientific literature referred herein is hereby incorporated by reference. The terms and definitions provided herein control, if in conflict with the terms and/or definitions incorporated by reference.

The singular forms “a,” “an,” and “the” include plural reference unless the context dictates otherwise.

The terms “approximately” and “about” mean to be nearly the same as a referenced number or value. As used herein, the terms “approximately” and “about” should be generally understood to encompass ±10% of a specified amount, frequency or value. With regard to specific values, it should be understood that specific values described herein for subject populations (e.g., the subject of the described clinical trial) represent mean values, unless otherwise indicated. Accordingly, aspects of the present disclosure requiring a particular value in a subject are substantially supported herein by population data in which the relevant value is assessed to be a meaningful delimitation of the subject population.

“Arm” or “study arm” used in the context of a clinical study or the design of a clinical study refers to a particular treatment regimen to be assessed in the clinical study and is usually characterized by a different dosage amount and/or dosing frequency taken by a predetermined set of patients in a dose range finding clinical study.

ABeta or AB or Aβ refers to beta amyloid peptide which forms plaques in the brains of AD sufferers; ABeta, AB or Aβ followed by the numbers 40 or 42 refers to the number of amino acids comprising the AB peptide. AB42 is associated most prevalently with plaque formation and therefore by inference brain pathology.

“BID” or “bid” means twice daily administration, when preceded by a quantity, it means that quantity is administered twice at different times in one day.

“Baseline” refers to a patient's physical and/or mental condition and measurements related thereto taken before administration of a study drug is begun.

“CSF” refers to cerebrospinal fluid.

“LTP” means long term potentiation and is used as an experimental model of memory formation. Bliss TV, et al., (1993). “A synaptic model of memory: long-term potentiation in the hippocampus”. Nature 361 (6407): 31-39.

“m-ITT” is define as the modified intent to treat population also referred to as the Full Analysis Set (FAS) and is those patients included based on the initial treatment intent (or study design) and not on the treatment that eventually was administered.

“PPS” is defined as the Per Protocol Set and is the number of subjects who completed the Phase II study and received at least 80% of the drug doses assigned to their dosage level (50, 1000, or 2000 mg BID).

“MMSE” is the Mini Mental State Exam which is a brief 30-point questionnaire test that is used to screen for cognitive impairment. It is commonly used in medicine to screen for dementia. It is also used to estimate the severity of cognitive impairment at a given point in time and to follow the course of cognitive changes in an individual over time, thus making it an effective way to document an individual's cognitive response to treatment. (See for example, Folstein M F et al, 1975, Journal of Psychiatric Research 12 (3): 189-98.)

“MRS” is magnetic resonance spectroscopy as applied in scanning images of the brain and measuring compounds in the brain.

“Placebo” refers to a pill or other dosage form that lacks the active pharmaceutical ingredient and which is administered in a double-blind clinical trial to the control arm patients, i.e., those patients who are not receiving the treatment being studied.

Scyllo-inositol is one of several endogenous stereoisomers of inositol. Myo-inositol (MI), which is the major endogenous inositol, plays an important role in osmoregulation and in phosphatidyl-inositol (PI) second messenger signaling. myo-inositol is found at ˜4-5 mM intracellular concentrations in adult brain, while scyllo-inositol concentrations are usually <1 mM. Scyllo-inositol, unlike myo-inositol, is not thought to be phosphorylated or directly involved in PI signaling.

Pre-clinical studies in AD have focused on beneficial effects of scyllo-inositol on memory and reasoning to the extent they can be measured in animal models and on more directly measureable outcomes such as maintenance or improvement of long term potentiation and reduction of plaque burden animals and cells. When given orally to a transgenic mouse model of AD, scyllo-inositol inhibits aggregation of amyloid β-peptide in the brain and ameliorates several AD-like phenotypes. In transgenic animals, scyllo-inositol reduced brain Aβ concentrations and plaque burden, preserved synaptic density, and improved learning deficits. Scyllo-inositol also appears to neutralize toxic effects of Aβ oligomers, including amelioration of oligomer-induced synaptic loss and dendritic densities, LTP inhibition, and memory/learning deficits.

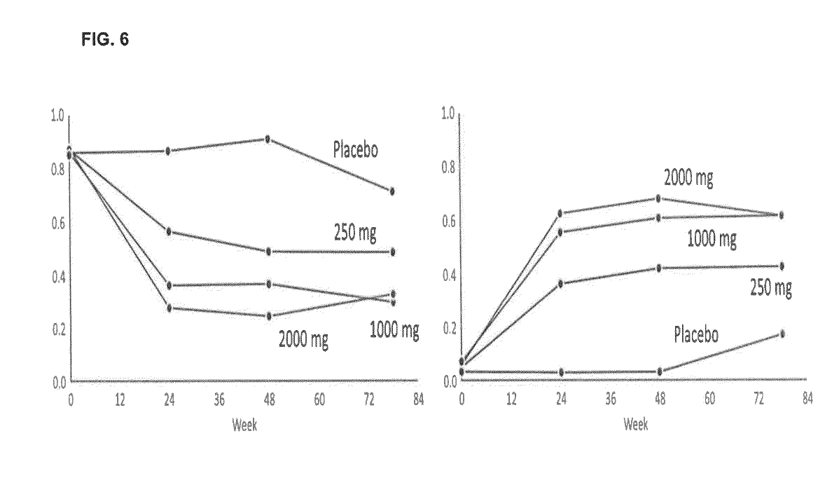

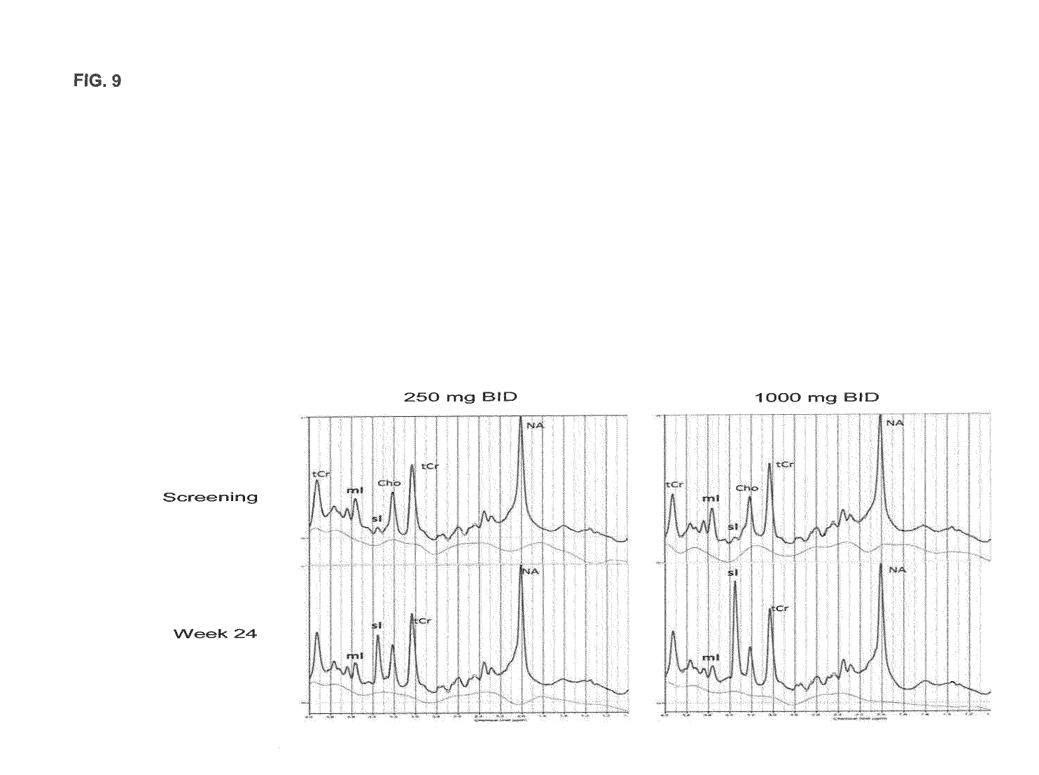

Scyllo inositol has completed phase II clinical studies for the treatment of cognitive symptoms of Alzheimer's Disease. The study included neuropsychological assessments using the NPI-12 item scale, as well as assessments of scyllo-inositol (SI) and myo-inositol (MI) brain levels using magnetic resonance spectroscopy (MRS). MRS data showed a dose-dependent increase of scyllo-inositol, and unexpectedly a corresponding dose-dependent decrease of myo-inositol levels (FIGS. 6 and 9); these changes are significant but sub-maximal at week 24, and reach maximal levels at about 48 to about 78 weeks. The maximal myo-inositol reduction measured at the 3 studied doses was 44%, 66% and 60% at the 250 mg, 1000 mg, and 2000 mg bid doses, respectively. While not being bound by any particular theory, scyllo-inositol is thought to competitively inhibit the active myo-inositol uptake by its transporter (Sodium-Sensitive myo-inositol transporter, Wiesinger, 1991, J Neurochem, 56(5):1698-704). The beneficial effects of scyllo-inositol on neuropsychiatric index outcomes seem to be based, at least in part, on the down-regulation of myo-inositol brain levels. The optimal range of myo-inositol reduction seems to be from about 20 to about 55%, or from about 25% to about 45%, or from about 25% to about 35% from baseline, while myo-inositol reductions at or above 60% appear to be associated with loss of clinical benefit and possibly with adverse CNS events.

The potential role of increased brain myo-inositol in brain dysfunction is further supported by MRS measurements in normally aged, MCI and AD. Elevated myo-inositol levels showed the strongest correlations with disease stage as measured by cognitive decline (myo-inositol levels were significantly higher in AD than MCI, and in MCI than in normal aged subjects). Thus the methods of lowering brain levels of myo-inositol by use of scyllo-inositol as disclosed herein are useful for down-regulating and maintaining more normal (i.e., clinically observed range in non-dementia, non-MCI or non-mild Alzheimer's controls) brain levels of myo-inositol in patients, such as dementia patients or Down's syndrome patients, where myo-inositol is elevated and in bipolar disorder where myo-inositol reduction has been demonstrated to have mood stabilizing therapeutic effects.

Based on the unexpected discovery, that administration of scyllo-inositol to a patient down regulates (i.e. reduces) the level of myo-inositol in the brain in such patients and delays the emergence, and lessens the degree, of neuropsychiatric symptoms (NPS), the present invention provides in one aspect a method of reducing the level of myo-inositol in a subject's brain comprising administering an effective amount of scyllo-inositol to the subject. In a particular embodiment, the subject is a human patient. In a particular embodiment, the patient has dementia. In a particular embodiment, the patient has Alzheimer's disease. In a particular embodiment, the patient has mild Alzheimer's disease with an MMSE of between 22 and 26. In a particular embodiment the patient has moderate Alzheimer's disease with an MMSE of between 16 and 21. In a particular embodiment, the patient has mild cognitive impairment. In a particular embodiment, the patient does not have dementia. In a particular embodiment, the patient does not have Alzheimer's disease. In a particular embodiment, the patient does not have mild cognitive impairment.

In another aspect of the invention, there is provided a method for treating a disease or condition in a patient wherein said disease or condition is mediated by high myo-inositol levels, comprising administering an effective amount of scyllo-inositol to the subject. In an embodiment, the subject is a human patient. In another embodiment, the disease or condition is bipolar disorder. In another particular embodiment the disease of condition is a subtype of a biopolar condition or disorder. In a particular embodiment, the disease or condition is type I bipolar disorder. In a particular embodiment, the disease or condition is type II bipolar disorder. In a particular embodiment, the disease or condition is a mixed bipolar disorder, rapid-cycling bipolar disorder, hypomania, cyclothymia, acute mania, drug-induced mania, or drug-induced hypomania. In another embodiment, the disease or condition is migraine headache. In another embodiment, the disease or condition is schizophrenia. In another embodiment, the disease or condition is agitation associated with Alzheimer's disease. In another embodiment, the disease or condition is agitation not associated with Alzheimer's disease. In another embodiment, the disease or condition is Down's syndrome. In another embodiment, the disease or condition is Down's syndrome wherein the patient is not suffering from A-beta associated neurodegeneration. In another embodiment, the disease or condition is migraine headache. In a particular embodiment, there is provided a method for preventing migraine headache in a patient, comprising administering an effective amount of scyllo-inositol to the subject.

In an aspect of the invention there is provided a method of treating a bipolar condition or disorder comprising administering to a subject in need thereof an amount of scyllo-inositol effective to reduce myo-inositol levels in the subject's brain. In embodiments of the invention the amount of myo-inositol is reduced by at least about 20%, 30%, 40%, 50% or 60%, in particular between about 20% to about 50%, from a baseline measurement taken prior to administration

In another aspect of the invention, there is provided a method for treating neuropsychiatric symptoms (NPS) in a subject comprising administering an effective amount of scyllo-inositol to the subject. In a particular embodiment, the NPS is bipolar disorder. In a particular embodiment, the disease or condition is erectile dysfunction. In another embodiment, the disease or condition is severe mood dysregulation. In another embodiment, the disease or condition is chronic pain syndrome. In another embodiment, the disease or condition is apathy. In another embodiment, the disease or condition is abberant motor behavior. In another embodiment, the disease or condition is loss of appetite. In another embodiment, the disease or condition is hallucinations. In another embodiment, the disease or condition is elation.

An “effective amount” or “therapeutically effective amount” of scyllo-inositol means the amount or dose of scyllo-inositol that provides the desired treatment or prophylactic effects in a patient, for example, reducing the severity or frequency of occurrence of the diseases and disorders herein. In an embodiment, an effective amount of scyllo-inositol is the amount required to reduce the level of myo-inositol in a patient's brain. In a particular embodiment, an effective amount of scyllo-inositol is the amount required to reduce the level of myo-inositol in a patient's brain to less than 60% from a baseline measurement prior to administration. In a particular embodiment, an effective amount of scyllo-inositol is the amount required to reduce the level of myo-inositol in a patient's brain by about 20% to about 55%, about 20% to about 50%, about 25% to about 45%, or about 25% to about 35% from a baseline measurement prior to administration. In a preferred embodiment, an effective amount of scyllo-inositol is the amount required to reduce the level of myo-inositol in a patient's brain by about 20% to about 50% from a baseline measurement prior to administration. An effective amount scyllo-inositol can vary according to factors such as the particular disease or disorder, the age, sex, and weight of the patient. A dosage regimen may be adjusted to provide the optimum therapeutic response. For example, several divided doses may be administered daily or the dose may be proportionally reduced as indicated by the exigencies of the therapeutic situation. In a particular embodiment, the amount of scyllo-inositol administered is about 1 mg to about 5000 mg. In a particular embodiment, the amount of scyllo-inositol administered is about 10 mg to about 2000 mg. In a particular embodiment, the amount of scyllo-inositol administered is about 100 mg to about 1500 mg per day. In a particular embodiment, the amount of scyllo-inositol administered is about 150 mg to about 1300 mg per day. In a particular embodiment, the amount of scyllo-inositol administered is about 200 mg to about 1200 mg per day. In a particular embodiment, the amount of scyllo-inositol administered is about 250 mg to about 1100 mg per day. In a particular embodiment, the amount of scyllo-inositol administered is about 300 mg to about 1000 mg per day. In a particular embodiment, the amount of scyllo-inositol administered is about 500 mg to about 1500 mg per day. In a particular embodiment, the amount of scyllo-inositol administered is about 600 mg to about 1300 mg per day. In a particular embodiment, the amount of scyllo-inositol administered is about 700 mg to about 1200 mg per day. In a particular embodiment, the amount of scyllo-inositol administered is about 800 mg to about 1100 mg per day. In a particular embodiment, the amount of scyllo-inositol administered is about 900 mg to about 1100 mg per day. In a particular embodiment, the amount of scyllo-inositol administered is about 1000 mg per day. In a particular embodiment, the amount of scyllo-inositol administered is 1000 mg per day. In a particular embodiment, the amount of scyllo-inositol administered is 1000 mg per day. In a particular embodiment, the amount of scyllo-inositol administered is about 500 mg per day. In a particular embodiment, the amount of scyllo-inositol administered is 500 mg per day. In a particular embodiment, the foregoing amounts of scyllo-inositol are administered once daily. In a particular embodiment, the foregoing amounts of scyllo-inositol are administered twice daily. In a particular embodiment, the amount of scyllo-inositol administered is about 250 mg twice daily. In a particular embodiment, the amount of scyllo-inositol administered is 250 mg twice daily. In a particular embodiment, the amount of scyllo-inositol administered is about 500 mg twice daily. In a particular embodiment, the amount of scyllo-inositol administered is 500 mg twice daily. In another particular embodiment, the foregoing amounts of scyllo-inositol are administered three times daily.

The methods of the invention also include co-administering other pharmaceutically active compounds prior to, following and contemporaneously with administration of scyllo-inositol. In a particular embodiment, scyllo-inositol is co-administered with therapeutic agents for treating neuropsychiatric disorders. In a particular embodiment, scyllo-inositol may be co-administered with or more additional therapeutic agents including without limitation beta-secretase inhibitors, gamma-secretase inhibitors, epsilon-secretase inhibitors, other inhibitors of beta-sheet aggregation/fibrillogenesis/ADDL formation (e.g. Alzhemed), NMDA antagonists (e.g. memantine), nonsteroidal anti-inflammatory compounds (e.g. Ibuprofen, Celebrex), anti-oxidants (e.g. Vitamin E), hormones (e.g. estrogens), nutrients and food supplements (e.g. Gingko biloba), statins and other cholesterol lowering drugs (e.g. Lovastatin and Simvastatin), acetylcholinesterase inhibitors (e.g. donezepil), muscarinic agonists (e.g. AFI 02B (Cevimeline, EVOXAC), AFI 50(S), and AF267B), anti-psychotics (e.g. haloperidol, clozapine, olanzapine), anti-depressants including tricyclics and serotonin reuptake inhibitors (e.g. SSRIs and SNRSs such as Sertraline and Citalopram HBr), statins and other cholesterol lowering drugs (e.g. Lovastatin and Simvastatin), immunotherapeutics and antibodies to A-beta (e.g. bapineuzumab), vaccines, inhibitors of kinases (CDK5, GSK3-alpha, GSK3-beta) that phosphorylate TAU protein (e.g. Lithium chloride), inhibitors of kinases that modulate A-beta production (GSK3-alpha, GSK3-beta, Rho/ROCK kinases) (e.g. lithium chloride and Ibuprofen), drugs that upregulate neprilysin (an enzyme which degrades A-beta); drugs that upregulate insulin degrading enzyme (an enzyme which degrades A-beta), agents that are used for the treatment of complications resulting from or associated with a disease, or general medications that treat or prevent side effects. In a particular embodiment, scyllo-inositol is co-administered with a mood stabilizer. In a particular embodiment, scyllo-inositol is co-adminstered with lithium e.g. lithium chloride. In a particular embodiment, scyllo-Inositol is co-administered with an antipsychotic Including without limitation risperidone (e.g, Risperidal®), quetiapine (e.g, Seroquel®) and ziprasidone (e.g, Geodone). In an embodiment, scyllo-inositol is co-administered with an antipsychotic selected from a phenothiazine, a thioxanthene, and in particular quetlapine, aripiprazole, haloperidol, olanzapine, clozapine, ziprasidone, chlorpromazine, thioridazine, mesoridazine, fluphenazine, perphenazine, prochlorperazine, trifluoperazine, thiothixine, molindone, loxapine, risperidone, aripirazole, and amisulpride. In an embodiment, scyllo-inositol is co-administered with an antipsychotic selected from abripiprazole, arisulpride, clozapine, quetiapine fumarate, haloperidol, loxapine succinate (Loxapac, Loxitane), clothiapine, metiapine, zotepine, molindone hydrochloride, olanzapine, paliperidone, pimozide, prochlorperazine (Compazine, Buccastem, Stemetil or Phenotil) risperidone, trifluoroperazine, zuclopenthixol (Clopixol), and combinations thereof. In an embodiment, scyllo-inositol is co-administered with a mood stabiliser drug selected from lithium, sodium valproate/valproic acid/divalproex, carbamazepine, lamotrigine, gabapentin, topiramate and tiagabine.

In an embodiment, scyllo-inositol is administered as a composition comprising the foregoing therapeutic agents. In an embodiment, the invention provides a pharmaceutical composition comprising scyllo-inositol and one or more second therapeutic agent. In some embodiments, the compositions comprise one or both active agents in subtherapeutic doses (e.g., amounts that are about 25%, 20%, 15%, 10%, 5%, 2%, 1% or less than a full dose). A composition may be in a form for consumption by a subject such as a pill, tablet, caplet, soft and hard gelatin capsule, lozenge, sachet, cachet, vegicap, liquid drop, elixir, suspension, emulsion, solution, syrup, aerosol (as a solid or in a liquid medium) suppository, sterile injectable solution, and/or sterile packaged powder.

A pharmaceutical composition comprising scyllo-inositol may also comprise a pharmaceutically acceptable carrier, excipient, or vehicle. A pharmaceutically acceptable carrier, excipient, or vehicle generally refers to a medium which does not interfere with the effectiveness or activity of an active ingredient and which is not toxic to the hosts to which it is administered. A carrier, excipient, or vehicle includes diluents, binders, adhesives, lubricants, disintegrates, bulking agents, wetting or emulsifying agents, pH buffering agents, and miscellaneous materials such as absorbants that may be needed in order to prepare a particular composition. Examples of carriers etc. Include but are not limited to saline, buffered saline, dextrose, water, glycerol, ethanol, and combinations thereof. The use of such media and agents for an active substance is well known in the art. Compositions and formulations for use in the present invention may be found in, for example, in Remington: The Science and Practice of Pharmacy, 21^{st} Ed., 2005; Martindale: The Complete Drug Reference, Sweetman, 2005, London: Pharmaceutical Press; Niazi, Handbook of Pharmaceutical Manufacturing Formulations, 2004, CRC Press; and Gibson, Pharmaceutical Preformulation and Formulation: A Practical Guide from Candidate Drug Selection to Commercial Dosage Form, 2001, Interpharm Press, which are hereby incorporated by reference herein.

Scyllo-inositol may be prepared according to various conventional synthetic or semi-synthetic techniques or isolated as a natural product from coconut palm. In a particular embodiment, scyllo-inositol is prepared according to the processes described in WO2005035774 and WO2011100670 the entirety of which are incorporated herein by reference.

### Example

In a Phase 2, parallel arm, dose-ranging, placebo-controlled, double-blind, multicenter trial in patients with mild to moderate AD (MMSE 16-26), scyllo-inositol was administered to study subjects in immediate release tablets twice daily at the dosage level set for each study arm or identical-appearing placebo tablets were administered in the control arm. The study showed no statistically significant benefit in the overall Mild and Moderate population on the co-primary cognitive and functional endpoints the Neuropsychological Test Battery and Alzheimer's Disease Cooperative Study—Activities of Daily Living Scale (NTB and ADCS-ADL respectively); but there were encouraging trends in the pre-specified group of Mild AD patients. The study included neuropsychological assessments using the NPI-12 item scale, as well as assessments of scyllo-Inositol (SI) and myo-inositol (MI) brain levels using magnetic resonance spectroscopy (MRS).

Study drug was administered as placebo or one of 3 doses of scyllo-inositol (SI): 250 mg, 1000 mg and 2000 mg, each dose given twice daily (bid) as capsules of either 250 mg or 1000 mg.

MRS data showed a dose-dependent increase of scyllo-inositol, and unexpectedly a corresponding dose-dependent decrease of myo-inositol levels (FIGS. 6 and 9), these changes are significant but sub-maximal at week 24, and reach maximal levels at about 48 to about 78 weeks. The maximal myo-inositol reduction measured at the 3 studied doses was 44%, 66% and 60% at the 250 mg, 1000 mg, and 2000 mg bid doses, respectively.

In this study, the 250 mg bid dose of scyllo-inositol showed the largest (positive) treatment effects on several clinical endpoints, compared to placebo and the 2 higher doses (data discussed in section below). The effect of the 250 mg dose on cerebrospinal fluid (CSF) Abeta42 reduction (˜27% compared to baseline) was statistically significant, but the higher doses seemed to provide further Abeta42 reduction. Despite the dose-dependent effects of scyllo-inositol on CSF Abeta42, the 2 high doses consistently produced smaller effects on clinical endpoints than the 250 mg dose. This observed dissociation between the dose-dependent effects on CSF Abeta42 and the clinically measured NPI outcomes supports the concept that scyllo Inositol clinical effects are mediated by two distinct mechanisms. These mechanisms include: 1. reduction of brain amyloid load and resulting synaptic toxicity which has previously been disclosed affect memory and reasoning/problem solving, but not NPS, and 2. down-regulation of brain myo-inositol levels with secondary salutary effects on neuronal function (Machado-Vieira et al. 2009). The only other MRS studies measuring in vivo brain levels of myo-inositol and scyllo-inositol after scyllo-inositol administration were in transgenic mice. (Choi, et al. 2010, Neuropharmacology, 59(4-5): 353-357). The Choi study showed statistically significant differential levels of scyllo-inositol in the frontal cortex and hippocampus of treated mice, with higher levels found in the hippocampus, and rather modest non significant myo-inositol reductions in both frontal cortex and hippocampus, although it is not possible to meaningfully compare the scyllo-inositol doses administered in the mice with the human doses reported herein. The details of the Phase 2 data are described below.

Summary of Phase 2 Clinical Data

The present study was a dose ranging study in Mild and Moderate AD patients (defined as having MMSE scores of 16-26 inclusive), and included 3 doses of scyllo-insoitol given bid (250 mg, 1000 mg, and 2000 mg) and placebo. The study was of 78 weeks duration and enrolled a total of 353 patients, and 351 received study drug (safety population). Due to safety findings in the 2 highest dose groups, these 2 groups were discontinued and the study's final efficacy analysis was based only on the 250 mg and placebo groups. The primary efficacy analysis was based on the overall population (mild and moderate), but the statistical analysis plan (SAP) Included subgroup analyses by disease severity, and defined Mild as patients with MMSE 23-26 and Moderate 16-22 inclusive. In the overall study population, total randomized to all 4 dose groups=353, total that received study drug=351 (safety population, at any dose). For placebo and 250 mg group, the overall m-ITT (modified intent-to-treat)=166, overall PPS (per protocol set)=96. The number of patients in each pre-specified subgroup is shown in Table 1.

TABLE 1
Distribution of Subjects in Placebo and 250 mg groups:
Mild (MMSE 23-26) and Moderate (MMSE 23-26) inclusive.

| | | | | |
|---|---|---|---|---|
| | Placebo | Placebo | 250 mg | 250 mg |
| Population | Mild | Moderate | Mild | Moderate |

| | | |
|---|---|---|
| Safety | 83 | 88 |

| | | | | |
|---|---|---|---|---|
| m-ITT | 35 | 47 | 36 | 48 |
| PPS | 22 | 25 | 24 | 25 |

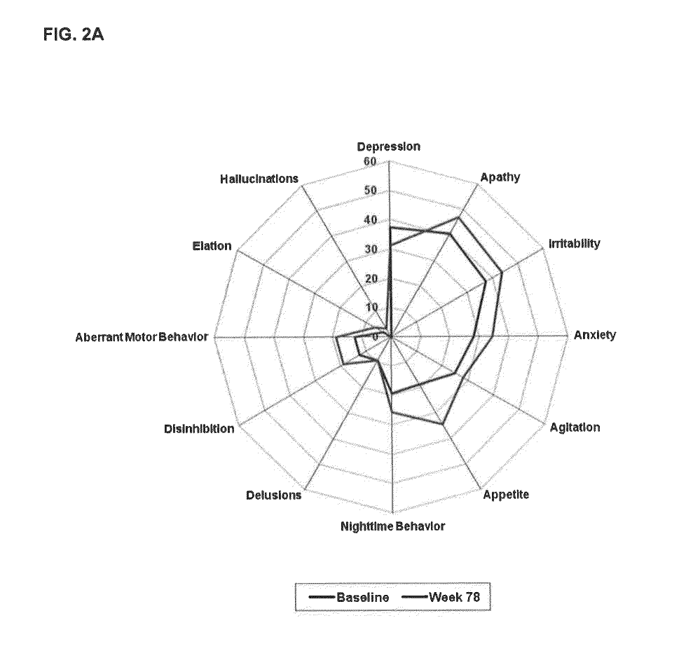

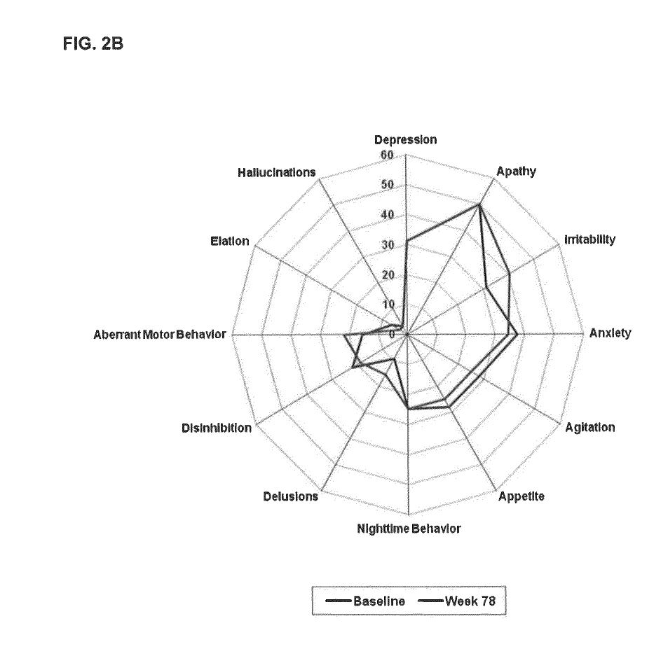

The two co-primary endpoints in the overall m-ITT population did not achieve statistical significance at end of study (week 78). However, in patients who completed the study and were at least 80% compliant with study drug (per protocol subjects); the NTB (neuropsychological test battery-cognitive endpoint) showed a numerical benefit in favor of the 250 mg group (FIG. 2). The magnitude of the 250 mg effect on the NTB, though not statistically significant, was clinically meaningful (0.15 or 40% benefit compared to placebo). In the moderate subgroup, there were no consistent negative or positive trends on the cognitive or functional clinical endpoints.

The pre-defined Mild subgroup (MMSE: 23-26) showed NTB differences in favor of drug that were also clinically relevant, and in the per protocol analysis reached statistical significance (see Table 1). The 2 functional outcomes measure Alzheimer's Disease Cooperative-Study-Activities of Daily Living Scale (ADCS-ADL) (co-primary endpoint) and Clinical Dementia Rating Scale-sum of boxes (CDR-SB) (secondary endpoint) also showed numerical differences in favor of the drug that were clinically meaningful (Table 2). Values for all clinical outcome measures were calculated so that a positive change from baseline indicates improvement, and a positive difference from placebo indicates drug benefit.

TABLE 2
Summary of Clinical Outcome Measures in 250
mg Mild Group (MMSE: 23-26 inclusive).

| | | ADCS- | |
|---|---|---|---|
| Measure | NTB | ADL | CDR-SB |
| Drug-placebo difference | | | |
| m-ITT (Placebo n = 35, 260 mg n = 36) | 0.2 | 2.26 | 0.87 |
| PPS (Placebo n = 22, 250 mg n = 24) | 0.4 | 2.25 | 0.95 |
| % effect compared to placebo | | | |
| m-ITT | 72% | 35% | 40% |
| PPS | 100% | 31% | 44% |
| p-value | | | |
| m-ITT | 0.11 | 0.43 | 0.19 |
| PPS | 0.007 | 0.46 | 0.20 |

A sensitivity analysis was performed post-hoc using a wider definition of the Mild sub-group (MMSE: 22-26 inclusive) with a larger sample size. In this sensitivity analysis, the positive effect of the 250 mg on the NTB score approached a significant trend (p<0.1) in the m-ITT analysis (Table 3), and was significant (p<0.05) in the PP analysis (data on file). This 250 mg group showed a numerically larger benefit on the ADCS-ADL; and the CDR-SB effect was also larger and approached a significant trend (p=0.1, Table 3). On this basis, all further analyses shown here are defined in the Mild and Moderate subgroups as patients having MMSE scores between 22-26 (Mild) and 16-21 (Moderate) inclusive.

TABLE 3
Summary of Clinical Outcome Measures in m-ITT 250
mg Mild Group defined by MMSE: 22-26 vs. 23-26.

| Measure (m-ITT population) | NTB | ADCS-DL | CDR-SB |
|---|---|---|---|
| Drug-placebo difference | | | |
| MMSE 23-26 (P: 35; 250 mg: 36) | 0.2 | 2.26 | 0.87 |
| MMSE 22-26 (P: 45; 250 mg: 43) | 0.2 | 3.15 | 0.97 |
| % effect compared to placebo | | | |
| MMSE 23-26 | 72% | 35% | 40% |
| MMSE 22-26 | 75% | 42% | 42% |
| p-value | | | |
| MMSE 23-26 | 0.11 | 0.43 | 0.19 |
| MMSE 22-26 | 0.07 | 0.22 | 0.10 |

Notably, the majority of patients in this study (˜90% across all dose groups) were already treated with symptomatic AD drugs; this included cholinesterase inhibitors, memantine, or both. In addition, ˜50% of all patients were also treated with psychoactive drugs, presumably for BPSD, and the proportion of treated subjects was similar between the placebo and 250 mg groups (data on file). Since the AD and psychoactive drugs may both have some effects on NPS, the effects of scyllo-inositol were apparent despite the background treatments. Since the profile of NPS varies by disease severity, the effects of scyllo-inositol on NPS are described separately for the Mild and Moderate subgroups.

The Effect of Scyllo-Inositol on the Neuropsychiatric Profile in Mild Ad (MMSE: 22-26)

The mean neuropsychiatric inventory (NPI) total scores for the Mild subgroup at baseline were 7.1 and 10.7 for the placebo and 250 mg groups (medians: 3.0 and 4.0), respectively. In the placebo group, the raw NPI scores increased (worsened) by 4.5 points over 78 weeks. In the m-ITT analysis, the 250 mg group showed only a numerical difference from placebo (˜2 points or 44% benefit compared to placebo), but the direction of treatment effect in the PP analysis was in the opposite direction (and neither was statistically significant). The low NPI scores at baseline make the demonstration of a potential treatment benefit in Mild patients difficult due to a “floor effect” on the NPI scale. Since Mild AD patients are known to progressively develop new NPS over time, a more appropriate analysis would be to evaluate treatment effects on the emergence of new NPS.

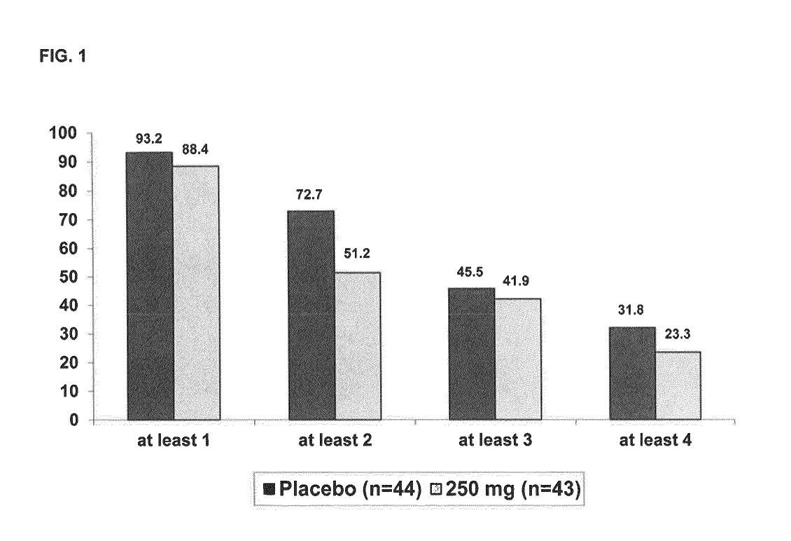

A potentially disease-modifying drug, by altering regional neuronal dysfunction, may prevent or delay the emergence of abnormal behaviors as quantified by the NPI outcomes. An NPI symptom is considered newly emergent when its score at baseline was 0, and became >0 at any subsequent visit. The effect of the 250 mg dose on the emergence of new NPS (for at least 1, 2, 3, or 4 new symptoms over the 78 weeks) is shown in FIG. 1. The proportion of patients who developed at least 2 new symptoms over 78 weeks was lower by 21.5% in the 250 mg group versus placebo (p<0.05).

In order to understand the nature of the most common NPS present in Mild patients prior to and at end of treatment, the prevalence of each NPI item is shown at baseline and at end of study (FIG. 2A). The prevalence of apathy, irritability, anxiety, agitation, appetite and sleep change (nighttime behaviors) increased in the placebo group at the end of study. In the 250 mg group, the prevalence of these symptoms remained stable (FIG. 2B), except for irritability which showed a 10% increase. A similar number of scyllo-inositol and placebo subjects in this Mild group reported irritability as an adverse event; and none of these subjects discontinued the study due to irritability (data on file). This suggests that the small increase in the prevalence of irritability in the 250 mg group did not pose a clinically significant finding.

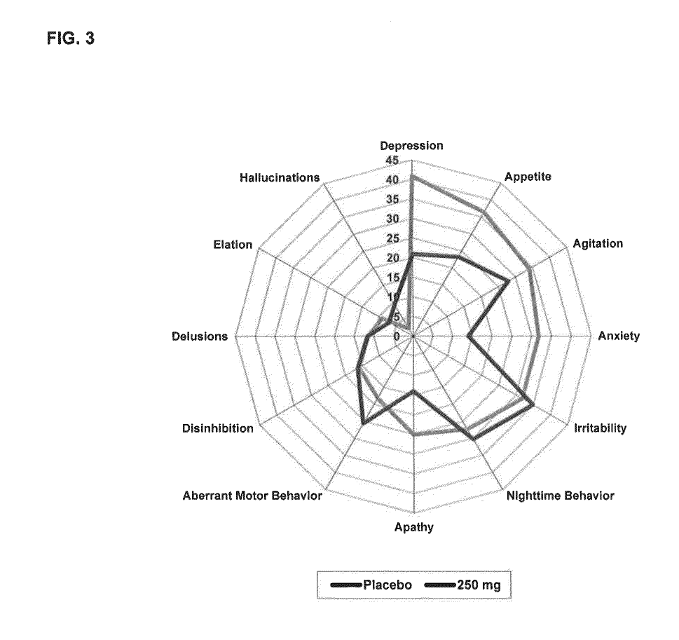

Some NPS are known to have a fluctuating course especially in the Mild stages of AD, and may therefore appear and disappear during the 78 weeks of the study. Therefore, assessment of the emergence of new NPS at any time during the study is also a factor (FIG. 3).

As shown in FIG. 3, depression and anxiety, as well as appetite change and agitation, are among the most commonly emergent symptoms; scyllo-inositol at 250 mg decreases the emergence of these symptoms and of apathy. Since patients frequently develop a variety of symptoms (symptom clusters) that are an expression of the same underlying cortical dysfunction, these clusters constitute specific behavioral syndromes.

Effect on Symptom Clusters:

The effect of scyllo-inositol on the time to emergence of the NPI symptom clusters was performed using a Kaplan-Meier survival analysis. Additionally, the effect of scyllo-inositol on decreasing the severity of symptoms (change from baseline of cluster scores) was also performed. Results of both analyses are shown in Table 4.

Each cluster score was the summation of its individual item scores, analyzed by a repeated measure mixed effect model to adjust for baseline differences. For this analysis, the clusters and their core symptoms were defined as: affective cluster (depression and anxiety), psychotic cluster (delusions and hallucinations), dys-executive or frontal cluster (Disinhibition and elation), hyperactivity cluster (core symptoms: aberrant motor and aberrant nighttime behavior), and apathy as a separate cluster.

TABLE 4
The effects of scyllo-inositol on the time to emergence of
NPS Syndromes in Mild AD (Kaplan-Meier Analysis), and on
Cluster Scores (Mixed Effect Repeated Measure Analysis).

| | Score | P value |
|---|---|---|
| | Difference | (Log Rank) |
| Cluster (Mild AD, m-ITT Population) | (MMRM) | (KM Analysis) |
| Affective: Depression and Anxiety | +0.65 | 0.079 |
| Psychotic: Delusions and Hallucinations | −0.29 | 0.555 |
| Apathy | −0.35 | 0.266 |
| Frontal: Elation and Disinhibition | +0.33 | 6.787 |
| Behavioral: Aberrant Motor/Nighttime | +0.05 | 0.390 |
| Behaviors | | |

As shown in Table 4, the most commonly emergent cluster in this Mild population is the affective syndrome. Using this basic definition of the affective cluster, scyllo-inositol 250 mg dose showed a trend of delaying the emergence of this syndrome (p<0.1). The scores for this cluster were also improved with treatment (0.65 improvement) compared to the placebo group.

The published studies that have examined symptom clusters in AD used variable methodologies, and the sample populations differed by source of referral (community versus clinic subjects), disease severity, and degree of medication use (of both AD and psychoactive drugs). As a result, a factor analysis based on the present study population was performed, since these data may more relevant for a clinical trial population. The factor analysis of the present study included all patients with baseline NPI data (n=351). In factor analyses, the Varimax procedure is the most commonly used method; and a sample size of ˜300-500 is considered to provide good reliability (Comrey and Lee, 1992, A first course in factor analysis, Hillsdale, N.J.: Eribaum). The Varimax rotation procedure revealed that the following factors loaded with a value equal to or greater than 0.4, and can thus be considered important factors in a cluster (data on file).

Affective:

Depression, anxiety, agitation, nighttime behavior, appetite changes and apathy.

Psychotic:

Delusions and hallucinations.

Frontal Lobe or Dys-Executive:

Elation/euphoria and Disinhibition.

The factor analysis of the present study therefore yielded similar results to published studies (discussed in prior section), with the exception of the “Affective Syndrome”. The main difference between the present results and prior studies is that appetite and sleep changes clustered within the affective syndrome, while irritability did not. This may in part reflect the fact that several studies utilized the 10-item NPI, which does not include appetite and sleep changes (for summary: Aalten et al. 2007, The Journal of Neuropsychiatry and Clinical Neurosciences, 19:50-56, Garre-Olmo et al. 2010, Quality of Life Research 19(3):445-53). The lack of clustering of “Irritability” with the affective symptoms may be related to 2 factors: 1. the high degree of psychoactive drug use in the Study AD201 population, which may have changed the relationship between irritability and depression/anxiety; and 2. The definition of “Mild” AD in this study identified a patient group that is milder than many prior studies (which usually define Mild as MMSE of 20-26). This latter possibility is supported by the results of Benoit et al. from the REAL-FR study (Benoit et al. 2003, Revue de Médecine Interne 24: 319s-324s), which set the lower MMSE for Mild at 21 and, similarly to the present findings, did not show clustering of irritability with affective symptoms. In the Factor Analysis, apathy had a borderline loading factor with the affective cluster (0.4). In most recent studies, Apathy has been considered an independent entity, but one previous study (Aalten et al. 2003, Dement Geriatr Cogn Disord 15: 99-105) found apathy to cluster with mood/affective symptoms.

The effect of scyllo-inositol 250 mg on the “affective syndrome” scores and time to emergence of this syndrome is shown in Table 5. The Affective Syndrome scores are shown by both mixed effect repeated measure analysis (MMRM), and by summary statistics. A positive difference between the drug and placebo (of the change from baseline) indicates drug benefit.

TABLE 5
The effects of scyllo-inositol on the emergence and
scores of the “Affective Syndrome” in patients
with Mild AD (MMSE: 22-26). This Affective Syndrome
was based on Study AD201 Factor Analysis.

| | | Score | |
|---|---|---|---|
| | Score | Difference | P value |
| Cluster (m-ITT | Difference | (Summary | (Log Rank) |
| Population) | (MMRM) | Statistics) | (KM Analysis) |
| Affective Cluster: | +1.36 | +3.47 | 0.118 |
| Depression, Anxiety, | | | |
| Agitation, Nighttime | | | |
| Behavior, Appetite | | | |
| Changes, Apathy | | | |

The effect of scyllo-inositol on the score changes by MMRM and summary statistics represents a 35% and 91% improvement compared to placebo group, respectively. The analysis of score changes by MMRM is a more conservative estimate since it adjusts for baseline imbalances between the 2 groups. The time to emergence of this more inclusive Affective Syndrome approached a significant trend despite the small sample size (Placebo n=31, 250 mg n=32).

The various analyses collectively support the efficacy of scyllo-inositol (250 mg bid) in decreasing the burden of psychopathology in Mild AD patients. These effects include decreasing the emergence and severity of 6 out of 7 commonly emergent symptoms.

To date, neither the AD symptomatic drugs nor the anti-depressant/anti-psychotic drugs have shown positive effects on both emergence and severity of such a wide range of affective symptoms. This profile of behavioral benefits, together with positive cognitive effects and good safety/tolerability in Mild AD patients, makes scyllo-inositol a treatment for this patient population.

The Effect of Scyllo-Inositol on the Neuropsychiatric Profile in Moderate AD (MMSE: 16-21).

The mean NPI total scores for the Moderate subgroup at baseline were 10.0 and 9.2 for the 250 mg and placebo groups respectively (medians: 7 for both groups). The NPI scores in the placebo group progressively worsened by ˜8 points over 78 weeks. The Moderate AD patients had a slightly higher burden of psychopathology than Mild patients at baseline, but the Moderate placebo group worsened more than the Mild placebo group (slightly less than double the rate of Mild patients). In moderate patients, the 250 mg group did not show significant differences from placebo in the m-ITT analysis. However, in compliant subjects who completed the study (PP analysis), the 250 mg group performed better than placebo by ˜4 points (representing ˜50% improvement). This degree of benefit, though not statistically significant, is clinically relevant.

Scyllo-inositol at 250 mg bid seemed to Improve the overall frequency and severity of NPS in Moderate AD patients. In order to understand which NPS contributed most to this drug effect, several analyses were performed. These analyses investigated the effect of scyllo-inositol on individual item scores, on cluster scores, and on emergence of new NPS.

The most prevalent NPS at baseline in the Moderate group were similar to the Mild subgroup except for irritability being replaced by nighttime behavior among the top 6 most prevalent NPS. In the moderate AD group, irritability was slightly less common, and aberrant motor behaviors and delusions were more common than in the Mild group (Table 6).

TABLE 6
Prevalence of Top 6 and bottom 6 NPS at baseline in Moderate
(MMSE: 16-21) and comparison to Mild Patients (22-26).

| NPI Items | Moderate | Mild |
|---|---|---|
| (Safety Population) | Patients | Patients |
| Apathy | 45% | 45% |
| Depression | 43% | 42% |
| Anxiety | 35% | 33% |
| Agitation | 28% | 28% |
| Nighttime Behaviors | 27% | 23% |
| Appetite Changes | 27% | 25% |
| Irritability | 25% | 31% |
| Aberrant Motor Behavior | 24% | 14% |
| Delusions | 18% | 8% |
| Disinhibition | 13% | 18% |
| Hallucinations | 8% | 3% |
| Elation/Euphoria | 8% | 6% |

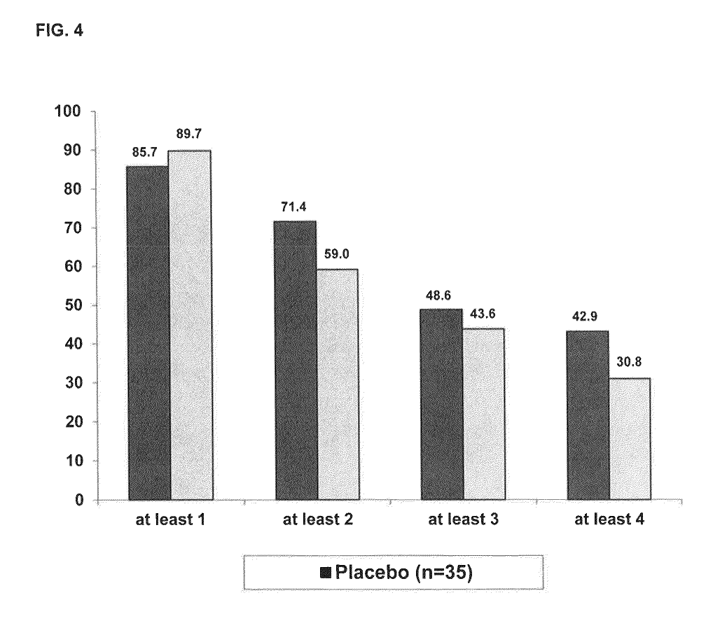

The emergence of new NPS (at least 1, 2, 3, or 4 new symptoms) in the Moderate group is shown in FIG. 4.

From FIG. 4, the proportion of patients who developed at least 2 or at least 4 new symptoms over 78 weeks was lower by 12.4% and 12.1% respectively, in the 250 mg group versus placebo, but none achieved statistical significance possibly due to the small sample size (p=0.33 and 0.34).

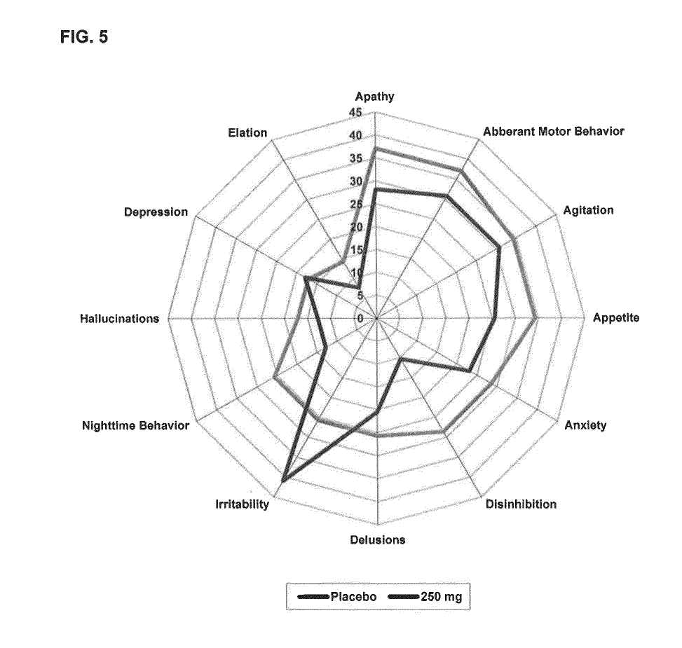

An assessment of the emergence of each NPI item was also performed and is shown in FIG. 5.

FIG. 5 illustrates that patients with Moderate AD have decreased emergence of most NPS. This effect is especially robust for disinhibition and sleep disturbances, which are more prominent at this stage of disease. Emergence of apathy, which is known to worsen with disease progression, also seems to be decreased by scyllo-inositol. Despite its low prevalence, the emergence of elation, which together with disinhibition is a core symptom of frontal dysfunction, was also decreased. Irritability, however, seems to be more prevalent with treatment. An evaluation of all cases where irritability was reported as an adverse event revealed that it is frequently associated with a prior history of mood disorders and/or insomnia. None of the irritability cases resulted in study discontinuation, suggesting that they were not clinically concerning.

Effects on Symptom Clusters:

The effect of scyllo-inositol on the time to emergence of NPI symptom clusters was performed using a Kaplan-Meier survival analysis. The symptom cluster that showed a trend to delayed emergence with treatment was the behavioral cluster in the PP analysis (aberrant motor and nighttime behaviors, p=0.051). The time to emergence of the frontal symptoms (disinhibition and elation) showed a possible weak trend (p=0.182), possibly due to the low prevalence of elation. The effects of scyllo-inositol on decreasing the severity of symptoms were also analyzed (change from baseline of cluster scores), using both MMRM and summary statistics (Table 7).

TABLE 7
The effects of scyllo-inositol on Cluster Scores in Moderate AD (by
Mixed Effect Repeated Measure Analysis and Summary Statistics).

| | Score Difference | |
|---|---|---|
| | (MMRM) | Score Difference |
| Cluster (Moderate AD) | m-ITT/PPS | m-ITT/PPS |
| Affective: | +0.94/+1.07 | −0.15/+0.06 |
| Depression and Anxiety | | |
| Psychotic: | −0.52/+1.32 | +0.34/+0.71 |
| Delusions and | | |
| Hallucinations | | |
| Apathy | +0.97/+0.14 | +1.09/+0.68 |
| Frontal: | +0.56/+0.68 | +0.48/+0.53 |
| Elation and Disinhibition | | |
| Behavioral: | +0.11/+1.42 | +1.76/+1.96 |
| Aberrant Motor, | | |
| Nighttime Behaviors | | |

Table 7 illustrates that the effect of scyllo-inositol in the Moderate AD group is driven by improvement of most NPS symptoms. The effects are most consistent on the aberrant behavioral cluster, on the affective cluster, and on apathy.

The effects of scyllo-inositol in the Moderate and Mild AD groups show some differences that may reflect the different profile of NPS progression at each disease stage, where Mild patients most accumulate new NPS, while Moderate patients have further worsening of existing NPS. The most prominent effect of scyllo-inositol in the Mild patients is on delaying emergence of new affective symptoms, even when the affective syndrome is defined broadly. In the moderate patients, the effect of scyllo-inositol is manifested by decreasing the worsening of various symptoms, including affective, behavioral, frontal, and apathetic symptoms.

Although the present study did not include patients with more severe dementia (MMSE below 16), it is reasonable to expect scyllo-inositol to show benefit in decreasing the severity of NPS in those patients, though the effect may be more prominent on the NPS that are more characteristic of the moderate to severe stage, such the psychotic symptoms of delusions, and hallucinations and of agitation and aggression. This is supported by a sensitivity analysis on the Moderate subgroup, where Moderate is defined by an MMSE bracket of 16-19, and therefore closer to the moderately severe stage than the 16-21 group analyzed above. In the “enriched” group of Moderate AD patients (MMSE 16-19), the effects of scyllo-inositol on the psychotic cluster, apathy, and aberrant behavior become more prominent.

From FIG. 6, the effect of scyllo-inositol at 1000 mg bid and 2000 mg bid seem to maximally saturate brain scyllo-inositol levels, and to cause a maximal decrease of myo-inositol levels of ˜60-66%, while the 250 mg bid dose led to a sub-maximal 44% decrease in myo-inositol levels, which is similar to the ˜30% reduction associated with therapeutic doses of Lithium when used in patients with bipolar disease. Although the present study did not include Down's syndrome patients, it is reasonable to expect scyllo-inositol to show benefit in decreasing the severity of NPS as a result in the change in myo-inositol. High myo-inositol levels are known to be present in Down's syndrome. (Shetty et al. 1996, Biochem J., 1; 313 (Pt 1):31-3; Shonk et al., 1995, Magnetic Resonance in Medicine, 33(6): 858-861; Beacher et al., 2005, Arch Gen Psychiatry. 62(12):1360-1365).

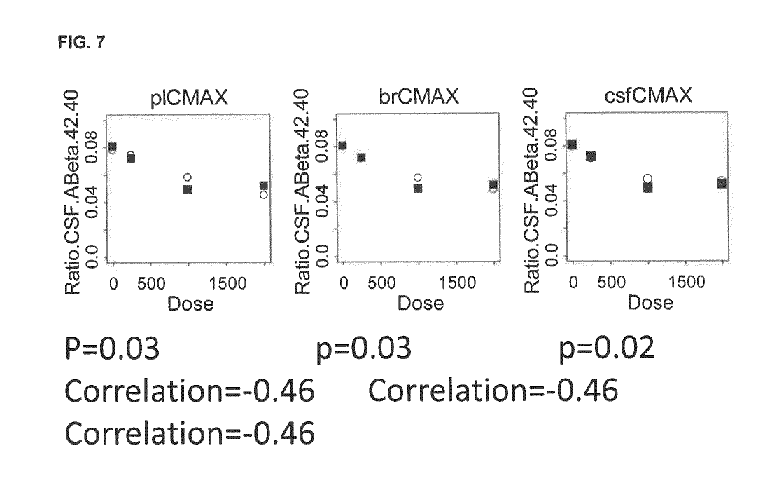

As provided in FIG. 7, despite the greater reduction of CSF Abeta42 by scyllo-inositol exposures corresponding to doses ≧1000 mg bid, these exposures did not show consistent benefits on the NPS in the symptom clusters. This supports the concept that regulation of myo-inositol brain levels plays an important role in the mediating the therapeutic benefits of scyllo-inositol

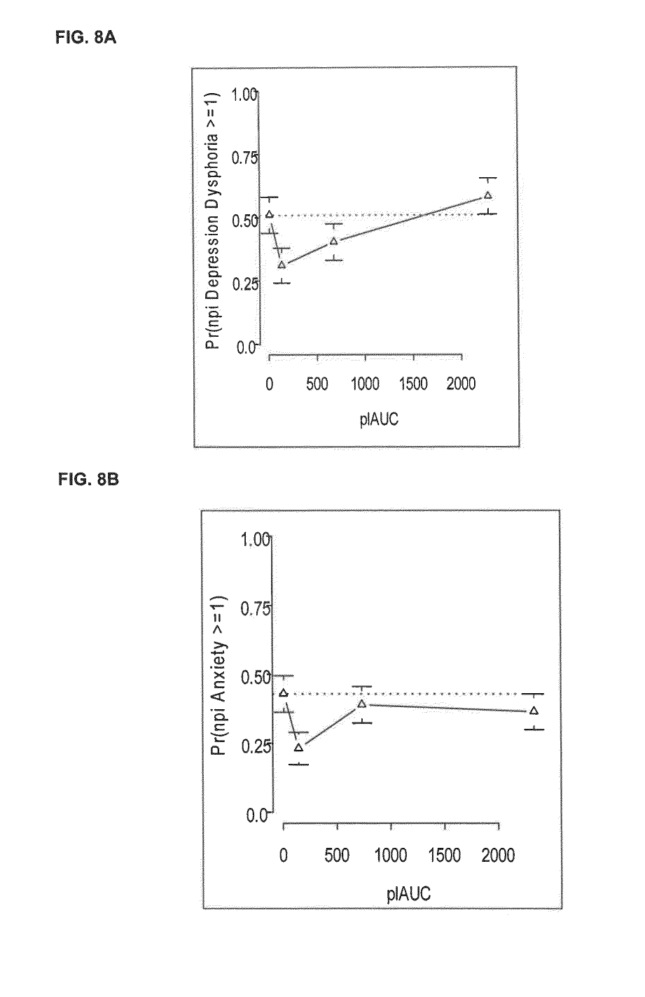

In view of FIGS. 8A and B, the plasma AUC corresponding to the 250 mg bid dose (second quartile) was associated with a significant decrease in emergence of depression and/or anxiety, while the highest exposures demonstrated no significant benefit. Of the doses tested, 250 mg bid dose led to a 27% reduction of Abeta42 CSF levels and to a 44% reduction of myo-inositol brain levels. This seems to be the optimal range of effects that mediates the clinical benefits on NPI.

In summary, scyllo-inositol exposures ≧1000 mg bid provide less cognitive and functional benefit than the 250 mg bid dose. These high exposures may be associated with an increase in neuropsychiatric adverse events. The 250 mg bid dose also demonstrated acceptable safety and good CNS tolerability in the elderly study population. The observations that the 250 mg bid exposures provide a positive clinical benefit/risk profile, and that the 250 mg bid dosing (500 mg total daily) both reduces CSF A▭42 beta reduction and sustainably down-regulates myo-inositol brain levels at therapeutically useful levels as demonstrated by the delay, prevention or lessening the NPI.

These data collectively help define the optimal therapeutic regimen of scyllo-inositol in the treatment of Alzheimer's disease and other dementias. These data also suggest that measurement of brain myo-inositol reduction may provide a way of predicting or determining optimal therapeutic dosing of scyllo-inositol in various dementia populations. Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

What is claimed is:

1. A method of reducing the level of myo-inositol in a subject's brain comprising administering an effective amount of scyllo-inositol to the subject.

2. A method for down-regulating myo-insoitol levels in a dementia, mild AD, MCI or bipolar disorder patient's brain comprising:
administering to the patient a therapeutically effective amount of scyllo-inositol for a therapeutically effective treatment period wherein the administration of scyllo-inositol reduces the myo-inositol levels in a patient's brain from a baseline measurement taken prior to administration.

3. The method of claim 1, wherein the effective amount of scyllo-inositol is from about 125 mg to about 900 mg per day.

4. The method of claim 3, wherein the scyllo-inositol is administered twice a day.

5. The method of claim 1, wherein administration reduces the patient's brain myo-inositol level by about 20% to about 50% from a baseline measurement prior to administration.

6. A method for delaying the emergence of at least two new neuropsychiatric symptoms in a patient comprising:
administering to the patient a therapeutically effective amount of scyllo-inositol over a therapeutically effective treatment period wherein over the treatment period, the administration of scyllo-inositol delays the emergence of at least two new neuropsychiatric symptoms compared to a baseline measurement prior to administration.

7. The method of claim 6, wherein the patient suffers from Alzheimer's disease dementia, fronto-temporal dementia, vascular dementia, Lewy Body dementia, or Down's Syndrome dementia.

8. The method of claim 7, wherein the Alzheimer's disease dementia is mild or moderate Alzheimer's.

9. The method of claim 6, wherein the therapeutically effective amount of scyllo-inositol is about 250 mg.

10. The method of claim 9, wherein the scyllo-inositol is administered twice a day.

11. A method for delaying the emergence of at least one neuropsychiatric symptom cluster in a patient with a dementia comprising:
administering to the patient a pharmaceutical composition comprising a therapeutically effective amount of scyllo-inositol over a therapeutically effective treatment period, wherein over the treatment period, the administration of scyllo-inositol delays the emergence of at least one neuropsychiatric symptom cluster compared to a baseline measurement prior to administration.

12. The method of claim 11, wherein neuropsychiatric symptom cluster is chosen from affective cluster, psychotic cluster, apathy, frontal lobe elation and disinhibition cluster, behavioral cluster, and any combination thereof.

13. The method of claim 11, wherein the therapeutically effective amount of scyllo-inositol is about 250 mg.

14. The method of claim 13, wherein the scyllo-inositol is administered twice a day.

15. The method of claim 11, wherein the dementia is chosen from Alzheimer's disease dementia, fronto-temporal dementia, vascular dementia, Lewy Body dementia, and Downs dementia.

16. The method of claim 11, wherein the dementia is moderate Alzheimer's disease and the at least one cluster is behavioral.

17. A method for reducing the severity of at least one neuropsychiatric symptom in a patient with dementia comprising:
administering to the patient a therapeutically effective amount of scyllo-inositol over a therapeutically effective treatment period, wherein over the treatment period, the administration of scyllo-inositol reduces the severity of at least one neuropsychiatric symptom from a baseline measurement prior to administration

18. The method of claim 17, wherein the at least one neuropsychiatric symptom is chosen from depression, anxiety, appetite change, agitation, nighttime behavior, delusions, hallucinations, apathy, disinhibition, sleep disturbances and elation.

19. The method of claim 17, wherein the dementia is chosen from Alzheimer's disease dementia, fronto-temporal dementia, vascular dementia, Lewy Body dementia, and Downs dementia.

20. The method of claim 17, wherein the therapeutically effective amount of scyllo-inositol is about 250 mg.

21. The method of claim 20, wherein the scyllo-inositol is administered twice a day.

22. A method for delaying the progression of at least one existing neuropsychiatric symptom in a patient with a dementia comprising:
administering to the patient a therapeutically effective amount of scyllo-inositol over a treatment period of at least 78 weeks, wherein over the treatment period, the administration of scyllo-inositol delays the progression of at least one existing neuropsychiatric symptom from a baseline measurement prior to administration.

23. The method of claim 22, wherein the dementia is chosen from Alzheimer's disease dementia, fronto-temporal dementia, vascular dementia, Lewy Body dementia, and Downs dementia.

24. The method of claim 23, wherein the dementia is moderate or severe Alzheimer's.

25. The method of claim 22, wherein the effective amount of scyllo-inositol is about 250 mg.

26. The method of claim 25, wherein the scyllo-inositol is administered twice a day.

27. Scyllo-inositol for use in reducing the level of, or down-regulating myo-inositol in a subject's brain, or in the preparation of a medicament for reducing the level of, or down-regulating myo-inositol in a subject's brain.

28. Scyllo-inositol for use in the treatment of neuropsychiatric symptoms in a subject or in the preparation of a medicament for treatment of neuropsychiatric symptoms in a subject.

29. Scyllo-inositol for use in delaying the emergence or reducing the severity of neuropsychiatric symptoms in a subject suffering from dementia, mild AD, MCI or bipolar disorder.

30. Scyllo-inositol as claimed in claim 29 wherein the neuropsychiatric symptoms are chosen from depression, anxiety, appetite change, agitation, nighttime behavior, delusions, hallucinations, apathy, disinhibition, sleep disturbances and elation.
